(19) United States
(12) Patent Application Publication
(10) Pub. No.: US 2015/0119976 A1
(43) Pub. Date: Apr. 30, 2015

(54) EXTRAVASCULAR DEVICE FOR LIMITING BLOOD FLOW ADJACENT AN ARTERIOVENOUS FISTULA

(71) Applicant: Abbott Cardiovascular Systems Inc., Santa Clara, CA (US)

(72) Inventors: Paul Consigny, San Jose, CA (US); Dariush Davalian, San Jose, CA (US)

(73) Assignee: Abbott Cardiovascular Systems Inc., Santa Clara, CA (US)

(21) Appl. No.: 14/066,330

(22) Filed: Oct. 29, 2013

(51) Int. Cl. A61B 17/12; A61L 31/14

(52) U.S. Cl. CPC A61B 17/12036; A61B 17/12131; A61L 31/148
USPC 623/1.38; 83/13

(57) ABSTRACT

A medical device that can be wrapped around a segment of an artery downstream of an arteriovenous fistula. The wrap, when used in this manner, creates a stenosis for reducing retrograde flow at the fistula. Sutures are positioned in holes present in the upper and lower halves or connecting ends of the wrap, after which the sutures are pulled to oppose the two sides in order to create a stenosis. When the surgeon is satisfied that the stenosis is appropriate, the sutures are tied in place.

### FIELD OF THE INVENTION

The present invention relates to implantable medical devices associated with the creation of, and/or the maturation of an arteriovenous (AV) fistula access structure for hemodialysis.

### BACKGROUND OF THE INVENTION

AV Fistula (a connection between an artery and a vein) are a desired access structure for the dialysis of kidney failure patients. FIG. 1 illustrates a matured portion of the vein near the artery, which acts as a re-usable cannula access site proximal the AV fistula.

About 42% of surgically created AV Fistula fail to mature; that is, the portion of the vein proximal the fistula fails to adapt physiologically to accommodate the higher arterial pressure. When this venous portion (or side of the AV fistula) matures, it becomes usable as a cannula access site for dialysis (FIG. 1). Maturation can take about 6 weeks from forming the fistula. Failure to mature and/or act as a good dialysis access site is most commonly the result of poor blood flow (low blood pressure/low blood flow rates) in the venous portion of the fistula. About 74% of these failures are salvaged by some form of intervention, followed by maturation of the venous side in another 6-8 weeks. The remaining about 11% of the cases are regarded as failures, which necessitates creating an AV Fistula at another site. The most common site of initial AV Fistula creation is the wrist. If a new AV Fistula is required, a new site proximal of the previous/failed site is chosen. Typically, there are 3 potential sites per arm.

Patients without a mature AV Fistula require some other, less desirable form of dialysis access for the standard 3 times a week dialysis regimen until a mature fistula is available. Additionally, about a third of mature fistula fail in a year. The health of kidney failure patients without a functioning mature AV Fistula deteriorates at a more rapid rate than those with one. Deteriorating health makes the subsequent creation of a functioning mature AV Fistula less probable, necessitating a significant number of interventions or access procedures resulting in poorer survival rates. Thus, a significant number of interventions and procedures may be avoided or significantly delayed, significant cost savings realized and the survival rate of dialysis patients significantly improved by decreasing the failure to mature rate of newly created AV fistula and by reducing the rate at which mature fistula fail.

AV fistula failure to mature is often tied to the development of a neointimal hyperplasia that occurs in areas adjacent to the arteriovenous anastamosis. The neointimal hyperplasia develops secondarily to turbulent blood flow at the anastamosis. A major contributor to this turbulence is believed to lie in the combination of retrograde and antegrade flow at the anastomosis—arterial flow coming towards the fistula not only from the upstream artery but also the downstream artery via collateral flow. See Bettinger, C J et al. Three-dimensional microfluidic tissue-engineering scaffolds using a flexible biodegradable polymer. Adv Mater 18: 165-169, 2005; and Guan, J et al. Preparation and characterization of highly porous, biodegradable polyurethane scaffolds for soft tissue applications. Biomaterials 26: 3961-71, 2005.

There are no known extravascular or perivascular devices available that can effectively and reliably assist a surgeon in limiting retrograde flow into the anastomosis. Accordingly, there is a need for a construct that can be effectively used by a surgeon to limit blood flow originating from the downstream artery, thereby reducing turbulence and the likelihood of anastomotic intimal hyperplasia and neointimal formation.

### SUMMARY OF THE INVENTION

The invention provides an extravascular or perivascular arteriovenous (AV) wrap intended for being placed over an artery downstream of a recently created anastomosis or AV fistula. The wrap may be adjusted by the surgeon when wrapped around the artery. The wrap is configured for reducing the inner diameter of the downstream artery, thereby reducing retrograde flow into the AV fistula. As such, the wrap provides an implantable device that can help reduce turbulence at the anastomosis and improve wall shear stress. By reducing turbulent blood flow at the fistula in this manner, it is believed there will be a concomitant reduction in the likelihood of a developing anastomotic intimal hyperplasia and neointimal formation.

It is envisioned that during the creation of the AV fistula, the surgeon will isolate a short segment of the downstream artery. According to one aspect of invention, a wrap, which may be bioresorbable, is then placed around the isolated segment of the downstream artery. In a preferred embodiment sutures are then positioned in holes present in the upper and lower halves or connecting ends of the wrap, after which the sutures are pulled to oppose the two sides in order to create a stenosis. When the surgeon is satisfied that the stenosis is appropriate, the sutures will be tied in place.

According to other aspects of invention, alternative connectors or connection methods include: (a) a wrap with opposing teeth and notches on the outer edges or connecting ends that lock the device in place, or (b) or a strip with teeth or chamfered edges that can be received in slots, similar to a cable wrap or tie type connection. The tie can be utilized to lock the wrap at progressively smaller diameters as the strip is being pulled through the slots. The objective of the designs is to reduce diameter of the device and artery sufficiently to create a hemo-dynamically significant stenosis in the downstream artery. The stenosis acts as a resistance that decreases retrograde blood flow and thereby reduces turbulence.

According to some embodiments, the wrap:
is constructed of bioresorbable polymers that have sufficient compliance to be able to allow bending for both the insertion of the artery and for collapsing the device to create the stenosis;
will have a lifetime that exceeds the time for the incorporation of the device into the artery and surrounding tissue by fibrosis;
depending on the desired absorption time, the wrap will be composed of polymers such as PLA, PLGA, poly(p-dioxanone, Tg is −16 C and will be very flexible at RT. In-vivo absorption 120 days), poly(trimethylene carbonate, Tg is −12 C and very flexible at RT. In-Vivo absorption is 2-3 years), copolymers of PLA PLGA, PLA polycaprolactone or PLA polyethylene oxide (PEO), an elastomer such as polyglycerol sebacate (PGS), PEG or polyurethane urea (PUU) elastomers; and/or
may elute a drug such as Everolimus to limit intimal thickening in the narrowed artery.

In accordance with the foregoing, there is a perivascular or extravascular wrap, medical device, method of use, method for making, or method for assembly of a medical device comprising such a wrap having one or more, or any combination of the following things (1)-(24):
(1) A wrap comprising a loop forming opposed connecting ends, and a distal and proximal ends, the loop is symmetric about one axis and asymmetric about a second axis orthogonal to the first axis; or the loop is symmetric about both axes.
(2) The loop ends are configured for being brought together and connected when the wrap is wrapped around an artery. The proximal and distal ends define a circumference about the same or less than the circumference of the wrap when the connecting ends are connected to each other and/or connecting ends portions of the loop contact each other.
(3) The wrap comprises a means for connecting the connecting ends, or connecting end means.
(4) The wrap includes means for nesting a first connecting end into a second connecting end. For example, one connecting end defines a peak that can nest in a valley of the opposing connecting end.
(5) A connector for connecting the ends of the loop may include sutures received in holes or wrapped around portions of the loop, or the connector is integral to the loop. When the connector is integral to the loop a first connecting portion and a second connecting portion is integral to the loop. When the first connecting portion is connected to the second connecting portion, the loop may be secured around the artery.
(6) The embodiments of FIG. 4-7.
(7) Connecting ends of the wrap are formed as undulating or curved ends configured to nest together. The undulations may be sinusoidal or zig-zag. The sinusoidal or zig-zag may describe ½ of a sinusoidal period, one sinusoidal period or 1.5 sinusoidal periods, which is shown in FIG. 4. The curves may be symmetric or asymmetric about a central axis of the loop (e.g., axis A in FIG. 4).
(8) A loop made from a unitary piece of material.
(9) A loop configured such that a distance between connecting ends is about 0 mm to about 2 mm, which corresponds to 45 degrees or about 12.5% circumference of a 5 mm diameter artery. The loop can include ties or sutures for securing the ends between the about 0 mm and 2 mm.
(10) The loop includes one or more supporting struts extending between the proximal and distal ends. The strut(s) may be equidistant from the connecting ends.
(11) The loop may be formed by a curved member or strut having a rectangular cross-section, or oval (rounded edges). The width of the member may be between about 1 to 3 mm and the thickness may be between about 0.1 to 1 mm. According to the disclosure the member may have a ratio of width to thickness of between 1 to 3, 1 to 30, 3 to 30, and 1 to 10.
(12) The wrap inner diameter (e.g., “C” in FIGS. 4-7) can range from between about 0.35 mm and 1.25 mm for a 25% diameter artery (“25% diameter” means the inner diameter of the artery is 25% of its inner diameter before placing the wrap 10), and between about 0.75 mm and 2.5 mm for a 50% diameter artery when the connecting ends are connected to each other.
(13) A loop where the connecting ends are straight or not curved.
(14) A loop where having connecting ends that may be selectively placed further or closer apart from each other, such as by using a cable-tie type connector.
(15) Depending on the desired absorption time, the wrap is composed of polymers such as PLA, PLGA, poly(p-dioxanone, Tg is −16 C and will be very flexible at RT. In-vivo absorption 120 days), poly(trimethylene carbonate, Tg is −12 C and very flexible at RT. In-Vivo absorption is 2-3 years), copolymers of PLA PLGA, PLA polycaprolactone or PLA polyethylene oxide (PEO), an elastomer such as polyglycerol sebacate (PGS), PEG or polyurethane urea (PUU) elastomers.
(16) A wrap with opposing teeth and notches on the outer edges or connecting ends that lock the wrap in place.
(17) A wrap having a strip with teeth or chamfered edges that are received in slots, similar to a cable wrap or tie type connection so that a wrap may be locked at progressively smaller diameters.
(18) The wrap has a body and connecting ends. The body may be substantially rectangular with ends configured to interlock with each other.
(19) An extravascular wrap, including a deformable loop configurable into a cylindrical-like body defining a space, such that when the loop is wrapped around an artery and formed into the body, a stenosis is formed in the artery; the loop including connecting ends configured for being brought together and held together when the loop is formed into the body.
(20) The apparatus of (19) or (21) through (24), in combination with one of, more than one of, or any combination in any order of the following list of things: wherein the loop is—symmetric about a first axis and asymmetric about a second axis orthogonal to the first axis, both axes passing through a center of the loop, and—the cylindrical-like body circumscribes the second axis; wherein the connecting ends are shaped to nest together when the body is formed; wherein the connecting ends are curved; wherein the connecting ends describe a sinusoidal shape; wherein the connecting ends including a slot or hole and a strip adapted for being engaged with the slot or hole; wherein the connecting ends include a cable tie like connector, or a male-female connector; further including at least one strut disposed within and connecting distal and proximal ends of the loop; wherein the at least one strut is straight; wherein the distance between the connecting ends is about 0 mm to about 2 mm, which corresponds to 45 degrees or about 12.5% circumference of a 5 mm diameter artery; wherein the has portions with cross section that are rectangular or oval; wherein the wrap includes first and second connector portions integral with the loop; wherein the wrap is made from a bioresorbable, bioabsorbable or biodegradable material; and/or wherein the loop further includes a distal and proximal end extending parallel to the first axis, wherein when the loop is formed into the cylindrical-like body the distal or proximal end define about a circumference of the body.
(21) A method of making a perivascular wrap, including cutting an loop from a tube or a sheet; wherein the loop is configured for forming a tubular body defining an inner diameter sized to create a stenosis at a segment of an artery.
(22) A method of treatment, including placing a wrap around a segment of an artery located downstream of a fistula; and reducing a diameter of the wrap to form a stenosis at the artery segment.
(23) An implantable medical device, including a deformable loop—symmetric about a first axis and asymmetric about a second axis orthogonal to the first axis, both axes passing through a center of the loop, and—including a first pair of ends adapted for being connected to each other to form a tubular body circumscribing the second axis.
(24) An apparatus, including a body including a loop, the loop including—a proximal and a distal end, and—a first and second connecting end, wherein the loop is adapted for being shaped into a tubular body when the first and second connecting ends are connected to each other, the tubular body having an inner diameter of between about 1.5 and 5.0 mm.

### INCORPORATION BY REFERENCE

All publications and patent applications mentioned in the present specification are herein incorporated by reference to the same extent as if each individual publication or patent application was specifically and individually indicated to be incorporated by reference. To the extent there are any inconsistent usages of words and/or phrases between an incorporated publication or patent and the present specification, these words and/or phrases will have a meaning that is consistent with the manner in which they are used in the present specification.

### BRIEF DESCRIPTION OF THE DRAWINGS

FIG. 1 is a side-view of the arm of a patient receiving dialysis. A fistula is shown.

FIG. 2 shows a wrap being wrapped around a portion of an artery located downstream of a fistula.

FIG. 3 shows the wrap of FIG. 1 secured around the artery. The wrap is symmetric about axis A and asymmetric about axis B.

FIG. 4 shows a side view of the wrap of FIG. 2.

FIG. 4A shows a close-up view of connecting ends of the wrap of FIG. 4.

FIG. 5 shows another embodiment of a wrap which has a strut.

FIG. 6 shows another embodiment of a wrap that has a variable wrap diameter.

FIG. 7 shows another embodiment of a wrap having a variable wrap diameter.

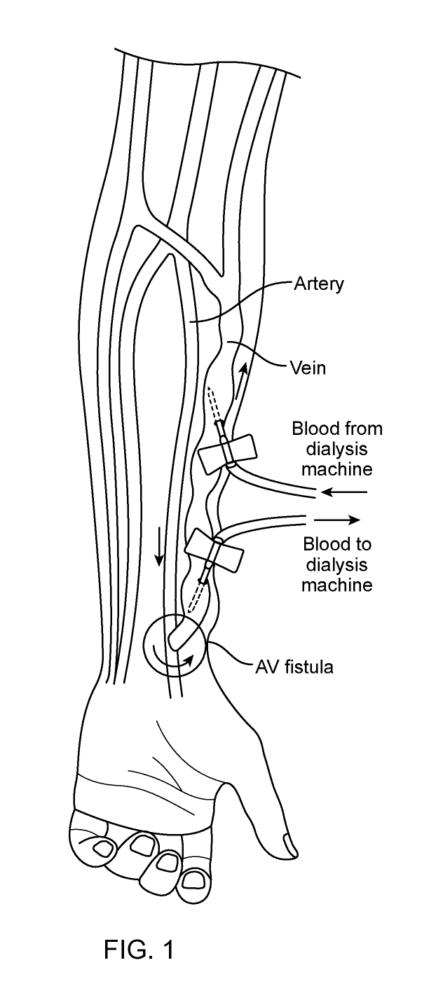

### DETAILED DESCRIPTION OF EMBODIMENTS

For purposes of this disclosure, the following terms and definitions apply:

When referring to a vein or artery prior to making a fistula, a “proximal end” refers to an end closest to the torso of the body, whereas a “distal end” refers to the end furthest from the torso of the body. In contrast, after the fistula is made, when referring to a medical device's intended location relative to a fistula or anastomosis, the terms “proximal” and “distal” are instead made with respect to the relative location of the fistula or anastomosis. Thus, for example, the end of a scaffold closest to the fistula will be called the “proximal” end and the end furthest from the fistula the “distal” end. Thus, generally speaking, prior to making the fistula the former terminology is used. And after the fistula is made “proximal” and “distal” always refers to a location relative to the fistula.

The terms “anastomosis” and “fistula” may be used interchangeably in this description. For purposes of the disclosure the two terms mean the same thing and refer to the arteriovenous (AV) type of anastomosis or fistula.

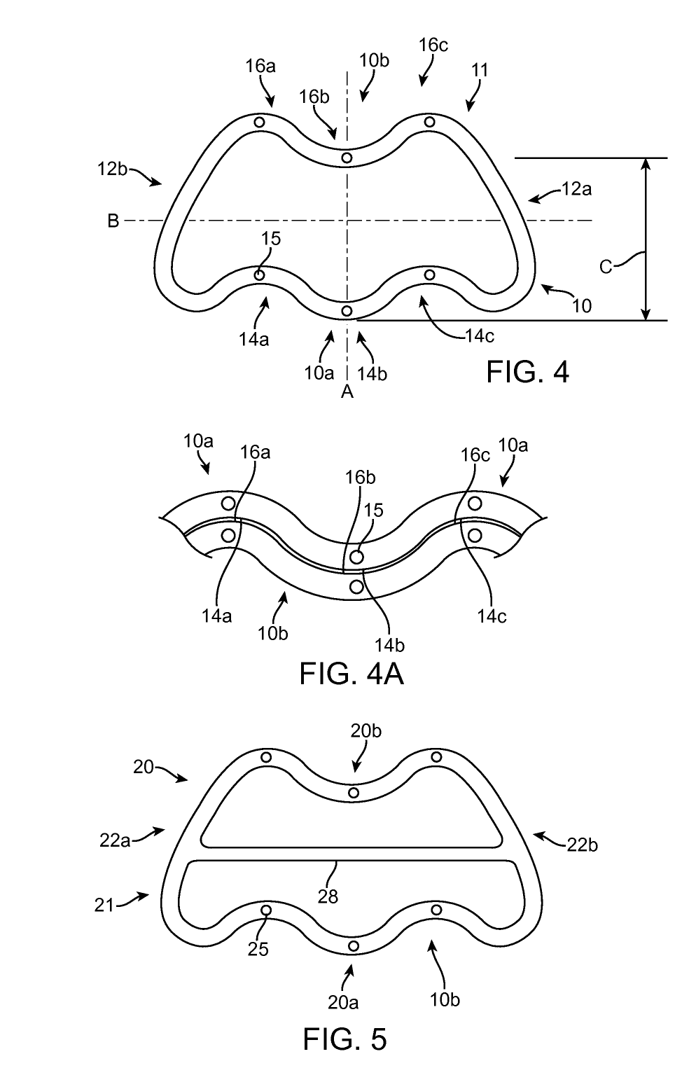

An “loop” is a deformable structure preferably made by cutting the loop (with ends connected) from a tube or flat sheet of material. A loop can be symmetric about one, two or no axis passing through the center of the loop. A loop has opposed connecting ends. The ends are brought together to circumscribe a space. When the loop is arranged in this manner it will be understood as forming a cylindrical-like or tube-like body defined substantially by the re-shaped loop having its connecting ends connected to each other. The loop 11 in FIG. 4 is symmetric about axis A but not axis B, which is circumscribed by the loop when the loop is formed into the tubular or cylindrical like body.

The term “about” means 20%, 15%, 10%, 5%, 4%, 3%, 2%, 1.5%, 1%, between 1-2%, 1-3%, 1-5%, or 0.5%-5% less or more than, less than, or more than a stated value, a range or each endpoint of a stated range, or a one-sigma, two-sigma, three-sigma variation from a stated mean or expected value (Gaussian distribution). It is understood that any numerical value, range, or either range endpoint (including, e.g., “about none”, “about all”, etc.) preceded by the word “about” in this disclosure also describes or discloses the same numerical value, range, or either range endpoint not preceded by the word “about”.

A “stent” is a permanent structure, usually comprised of a metal or metal alloy, generally speaking, while a scaffold will refer to a structure comprising a bioresorbable polymer and capable of radially supporting a vessel for a limited period of time, e.g., 3, 6 or 12 months following implantation. It is understood, however, that the art sometimes uses the term “stent” when referring to either type of structure.

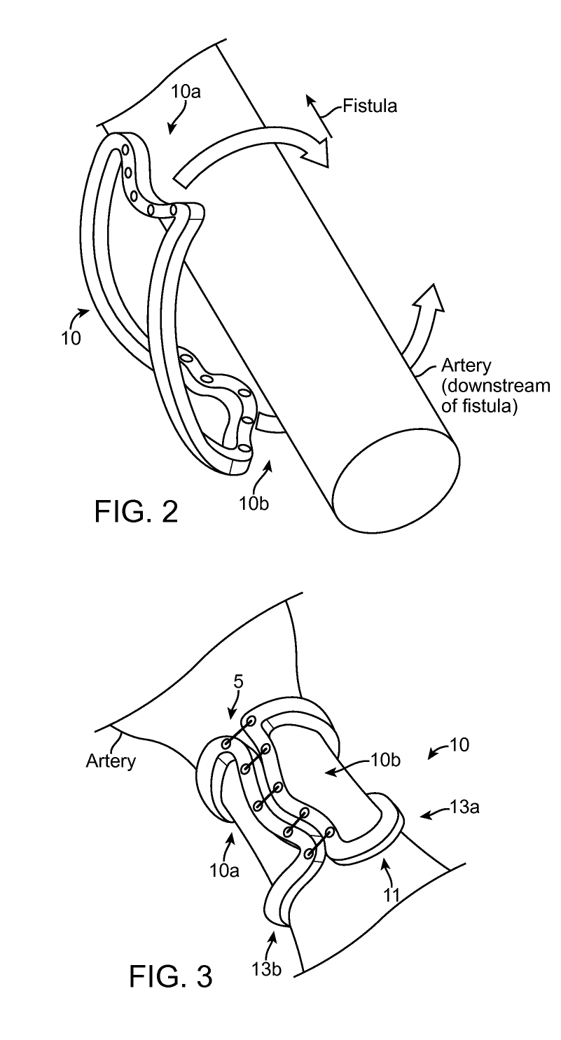

Referring to FIGS. 2 and 3, there is an extravascular wrap 10 placed around an artery at a location downstream of a recently formed arteriovenous (AV) fistula. The wrap 10 has a first end 10a and a second end 10b. The wrap 10 is flexible, enabling it to be easily wrapped around the artery.

When the desired fitting is found the ends 10a and 10b are held together by, e.g., sutures 5. With the wrap 10 in place (FIG. 3) the lumen of the artery is narrowed. In some embodiments the wrap is formed so that the artery lumen is narrowed by 75%; that is, the lumen diameter is 25% its diameter before the wrap 10 is applied. Preferably the wrap 10 may be wrapped to narrow the lumen diameter within a range of lumen diameters when the sutures are placed. For example, the wrap ends 10a and 10b may be held in place further or closer together by sutures 5, thereby forming a narrowed lumen having a lumen diameter of between about 20% to 50% of the lumen diameter before the wrap 10 was placed (FIG. 2). The wrap 10 is intended to form a stenosis of desired diameter, suitably chosen to affect the downstream flow and/or pressure to reduce backflow, turbulence or circular motion of blood at the anastomosis.

A wrap according to the disclosure may be made from a bioresorbable or bioabsorbable polymer. Depending on the desired absorption time, the wrap may be composed of polymers such as PLA, PLGA, poly(p-dioxanone, Tg is −16 C and will be very flexible at RT. In-vivo absorption 120 days), poly(trimethylene carbonate, Tg is −12 C and very flexible at RT. In-Vivo absorption is 2-3 years), copolymers of PLA PLGA, PLA polycaprolactone or PLA polyethylene oxide (PEO), an elastomer such as polyglycerol sebacate (PGS), PEG or polyurethane urea (PUU) elastomers.

A wrap according to the disclosure may be formed from a tube, which was formed by extrusion or injection molding. Or the wrap may be made from a flat sheet of material. In either case the wrap may be laser cut into the desired shape. Examples of a tube forming process suitable for making a wrap according to the disclosure is described in US20120073733 (docket no. 104584.14); US2011/0066222 and US20100244304 (docket no. 62571.330).

FIGS. 5-7 show four embodiments of a perivascular or extravascular wrap according to the disclosure. The wrap of these embodiments each include a body that is a flexible loop capable of being wrapped around an artery to form the stenosis. The loop has a proximal end, distal end and connecting ends. The connecting ends are configured for being brought towards each other and connected as the loop is wrapped around the artery (e.g., as shown for wrap 10 in FIGS. 2-3).

It will be understood that these embodiments are not mutually exclusive of each other. Rather, embodiments according to the disclosure may be a combination of the features shown in the drawings, which are intended only as exemplary of wraps. For example, the connectors 45a, 45b for the connecting ends for the wrap of FIG. 7 may be used in place of suture holes 15 in FIG. 4.

FIG. 4 shows a planar view of the wrap 10 from FIGS. 2-3. The wrap body 11 has a proximal end 12b, distal end 12a and connecting ends 10a, 10b. The connecting ends 10a, 10b may include matching holes for threading through a suturing thread to hold the ends 10a, 10b together. In the illustrated embodiment there are three holes 15 at each of the connecting ends. Or the body 11 may not have the pre-made holes. In this case the suturing threads 5 (FIG. 3) can be wrapped around the ends and secured. Preferably, the body 11 forms a loop.

The connecting ends 10a, 10b may each form an undulating and complimentary portion of the loop 11 so that when brought together (e.g., as shown in FIG. 3) peaks of one end fit within a valley of the other end. For example, the ends 10a, 10b may each be described as sinusoidal and in-phase with each other so that peaks of one end nestle into valleys of the other end, as depicted in FIG. 4A.

Referring to FIGS. 4 and 4A, end 10b forms a valley 16b centrally and peaks 16a, 16c to the left and right, respectively, of the valley 16b. End 10a forms a peak 14b centrally and valleys 14a, 14c to the left and right, respectively, of the peak 14b. When the ends 10a, 10b are joined, such as when wrap 10 is being wrapped around the artery (FIG. 3) the peak 14b is received within the valley 16c and the peaks 16a, 16c are received within the respective valleys 14a, 14c. In FIG. 4 the ends 10a, 10b each describe one and one-half periods of a sinusoid symmetric about axis A. Thus, the sinusoid is symmetric about axis A, which passes through both the peak 14b and valley 16b.

In alternative embodiments connecting end(s) describing a sinusoid may be asymmetric about the axis A, in which case there may be one or more peaks on one side and one or more valleys on the opposite side (axis A passes between the peak and valley). The ends may alternatively describe %, one or more than one period of a saw tooth or square tooth wave, so that, e.g., square teeth fit within opposing rectangular gaps on the opposite connecting end when et connecting ends are mated or joined. Thus, in the embodiments, the ends 10a, 10b are configured to have complimentary shapes enabling an interlocking or nesting between the ends as this will help secure the wrap in place around the artery. This shapes may be symmetric or asymmetric about axis A.

The loop 11 may be formed by strut portions having a rectangular cross-section, or ovid (rounded edges). The width of the strut portions may be between about 1 to 3 mm and the thickness may be between about 0.1 to 1 mm. According to the disclosure the strut portions may have a ratio of width to thickness of between 1 to 3, 1 to 30, 3 to 30, and 1 to 10.

The dimension “C” in FIG. 4 refers to the inner diameter of the wrap 10 when the ends are connected and abut each other (FIG. 3). This diameter corresponds to the desired narrowing of the artery when the wrap 10 is wrapped around the artery to form the stenosis. C is measured from the edge of end 10a to the edge of the opposing end 10b; e.g., the distance between the outer edge at 16b to the outer edge at 14b.

For normal arteries of the wrist where AV fistula are made the artery outer diameter is between about 1.5 mm and 5.0 mm. As such, C can range from between about 0.35 mm and 1.25 mm for a 25% diameter artery (“25% diameter” means the inner diameter of the artery is 25% of its inner diameter before placing the wrap 10) and between about 0.75 mm and 2.5 mm for a 50% diameter artery.

FIG. 4 shows a wrap 20 that is the same as wrap 10 except that a supporting strut 28 extends between ends 20a, 20b and connects proximal and distal ends 22b, 22a of the body 21, which in this case is also an loop 21. The strut may be located equidistant from ends 20a, 20b. There may be at least one or more struts 28 extending between ends 22a, 22b.

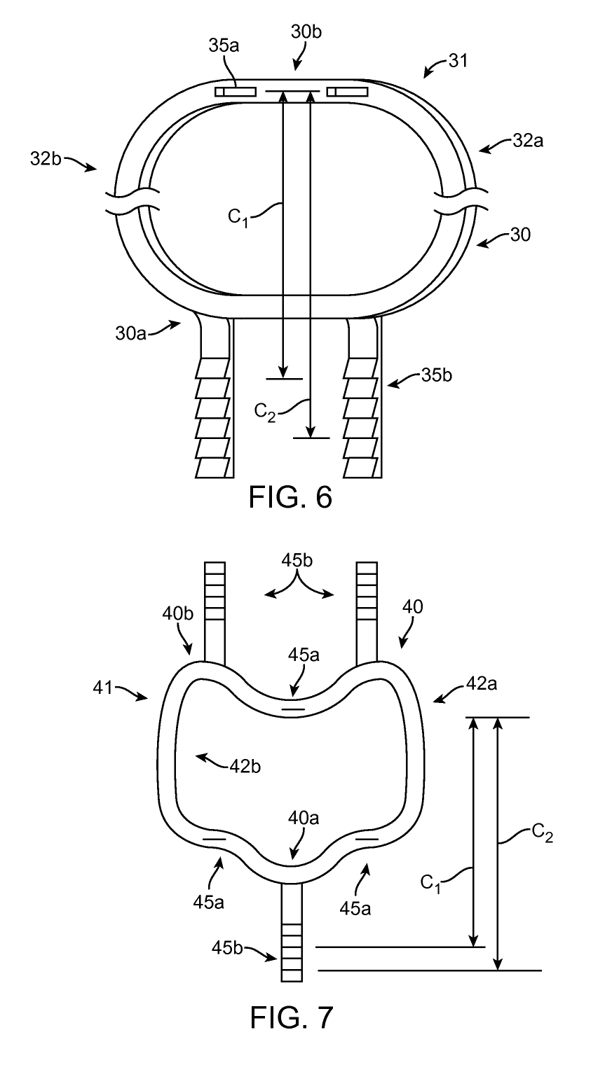

FIG. 6 shows an alternative. Here there is a loop 31 where the connecting ends 30a, 30b include a portion of the loop 31 and strips 35b. The ends 30a, 30b are about straight or not curved. The loop 31 is symmetric about two orthogonal axes A and B. Additionally, the suture holes 15 are replaced by rectangular slots 35a at end 30b sized for forcibly receiving ends 35b of the strips 36b disposed at the opposed end 30a (i.e., an elastic or inelastic deformation occurs with one or both of structure 35a, 35b when strip 35b is placed into slot 35a). The strip 35b have formed thereon a plurality of chamfered edges for providing an adjustable diameter wrap 30, as indicated by the variable C distance (C1, C2 which refer to the different inner diameters for forming different artery diameters such as 25% or 50%). The connecting ends strip and slots 35a, 35b may be thought of as a cable-tie type connector for wrap 30 (as opposed to holes 15 for receiving suturing thread in the case of wrap 10). The cable tie ends 35b may be cut from a tube or sheet of material with the loop, or welded or glued to the loop end 30a after forming the loop 30.

FIG. 7 shows an embodiment of a wrap 40 where the loop 41 has proximal and distal ends 42b, 42a and end portions 40a, 40b of connecting ends. In the case of ends 40a, 40b they are curved as in FIG. 4 but the curved shape is different. The curve is symmetric about the axis A and describes a single peak and opposed valley, or single peak with greater amplitude than adjacent left and right peaks or valleys. In this case the curved shape may be described by ½ a period of a sinusoid with the peak (or valley) centered about the axis A. Thus end 40a forms a single peak and valley and end 40b forms a single peak and valley.

In FIG. 7 there are three strips 45b received through or connected to matching connectors 45a. The trips 45b and matching connectors 45a may describe a cable-tie like connector, or a male and female type connection. For example, the strip 45b has a plurality of rounded male-type protuberances extending along the strip 45b that may be snap-fit into a female holes 45a. As in the case of FIG. 5 the wrap 40 may be wrapped around and form different diameters as needed.

In other embodiments a wrap has a loop and opposed connecting ends, where at one connecting end there is a strip that forms a u-shaped channel that may be snap-fitted over the opposing of the opposing end of the loop.

The above description of illustrated embodiments of the invention, including what is described in the Abstract, is not intended to be exhaustive or to limit the invention to the precise forms disclosed. While specific embodiments of, and examples for, the invention are described herein for illustrative purposes, various modifications are possible within the scope of the invention, as those skilled in the relevant art will recognize.

These modifications can be made to the invention in light of the above detailed description. The terms used in the claims should not be construed to limit the invention to the specific embodiments disclosed in the specification. Rather, the scope of the invention is to be determined entirely by the claims, which are to be construed in accordance with established doctrines of claim interpretation.

What is claimed is:

1. An extravascular wrap, comprising:
a deformable loop configurable into a cylindrical-like body defining a space, such that when the loop is wrapped around an artery and formed into the body, a stenosis is formed in the artery;
the loop including connecting ends configured for being brought together and held together when the loop is formed into the body.

2. The wrap of claim 1, wherein the loop is
symmetric about a first axis and asymmetric about a second axis orthogonal to the first axis, both axes passing through a center of the loop, and
the cylindrical-like body circumscribes the second axis.

3. The wrap of claim 2, wherein the connecting ends are shaped to nest together when the body is formed.

4. The wrap of claim 1, wherein the connecting ends are curved.

5. The wrap of claim 4, wherein the connecting ends describe a sinusoidal shape.

6. The wrap of claim 1, wherein the connecting ends including a slot or hole and a strip adapted for being engaged with the slot or hole.

7. The wrap of claim 6, wherein the connecting ends include a cable tie like connector, or a male-female connector.

8. The wrap of claim 1, further including at least one strut disposed within and connecting distal and proximal ends of the loop.

9. The wrap of claim 8, wherein the at least one strut is straight.

10. The wrap of claim 1, wherein the distance between the connecting ends is about 0 mm to about 2 mm, which corresponds to 45 degrees or about 12.5% circumference of a 5 mm diameter artery.

11. The wrap of claim 1, wherein the has portions with cross section that are rectangular or ovid.

12. The wrap of claim 2, wherein the wrap includes first and second connector portions integral with the loop.

13. The wrap of claim 12, wherein the wrap is made from a bioresorbable, bioabsorbable or biodegradable material.

14. the wrap of claim 13, wherein the loop further includes a distal and proximal end extending parallel to the first axis, wherein when the loop is formed into the cylindrical-like body the distal or proximal end define about a circumference of the body.

15. A method of making a perivascular wrap, comprising:
cutting an loop from a tube or a sheet;
wherein the loop is configured for forming a tubular body defining an inner diameter sized to create a stenosis at a segment of an artery.

16. The method of claim 15, wherein the wrap is made from a bioresorbable, bioabsorbable or biodegradable material.

17. An implantable medical device, comprising:
a deformable loop
symmetric about a first axis and asymmetric about a second axis orthogonal to the first axis, both axes passing through a center of the loop, and
including a first pair of ends adapted for being connected to each other to form a tubular body circumscribing the second axis.

18. The medical device of claim 17, wherein the ends are configured for being selectively spaced over a circumferential distance of between about 0 mm and 2 mm from each other when the tubular body is formed.

19. The medical device of claim 17, further including means for connecting the ends including a connector being integral with the loop.
